SEQUENCE LISTING (7 sequences)

SEQ ID NO: 1 - protein, 373 aa, Clostridium sp.
YYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFIENSGQNIERNPALQKLSSESVVDLFTKVCLRLTKNS

SEQ ID NO: 2 - protein, 1274 aa, Clostridium sp.
WPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 3 - protein, 40 aa, Unknown
  source: /note=“Description of Unknown GHRH sequence”
MPLWVFFFVILTLSNSSHCSPPPPLTLRMRRYADAIFTNS

SEQ ID NO: 4 - protein, 440 aa, Clostridium sp.
PVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVK

SEQ ID NO: 5 - protein, 421 aa, Clostridium sp.
PKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIR

SEQ ID NO: 6 - protein, 421 aa, Clostridium sp.
PKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGADGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIR

SEQ ID NO: 7 - protein, 829 aa, Clostridium sp.
KSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

(19) United States
(12) Patent Application Publication
(10) Pub. No.: US 2021/0128702 A1
(43) Pub. Date: May 6, 2021

(54) USE OF A BOTULINUM TOXIN AGENT FOR TREATING PLASMA CELL DISORDERS

(71) Applicant: Dana-Farber Cancer Institute, Inc., Boston, MA (US)

(72) Inventors: Giada Bianchi, Boston, MA (US); Kenneth Anderson, Wellesley, MA (US)

(21) Appl. No.: 16/635,714

(22) PCT Filed: Aug. 13, 2018
(86) PCT No.: PCT/US2018/046539

§ 371 Date: Jan. 31, 2020

Related U.S. Application Data
(60) Provisional application No. 62/544,611, filed on Aug. 11, 2017.

(51) Int. Cl. A61K 38/48; A61K 31/69; A61K 38/07; A61K 31/407; A61K 38/05; A61K 38/06; A61P 35/00

(52) U.S. Cl. CPC A61K 38/4893; A61K 31/69; A61K 38/07; A61K 48/00; A61K 38/05; A61K 38/06; A61P 35/00; A61K 31/407

(57) ABSTRACT

This disclosure relates to compositions and methods of treating plasma cell disorders and/or disorders associated with protein secretion, production, or deposition.

### TECHNICAL FIELD

This disclosure relates to compositions and methods of treating plasma cell disorders and/or disorders associated with protein secretion, production, or deposition.

### BACKGROUND

Multiple myeloma (MM) and amyloid light-chain amyloidosis (AL) are incurable plasma cell (PC) disorders characterized by aberrant proliferation of a clonal plasma cell and increased synthesis/secretion of a clonal immunoglobulin (paraprotein) and/or free light chains (FLC).^{1, 2} In MM, the etiology of symptoms/signs is related to excessive proliferation of MM cells, excessive paraprotein/FLC secretion/deposition, or both. In AL, the amyloidogenic FLC deposit in organized β sheets in target organs such as heart, kidney, or nerves, leading to progressive organ failure and eventually death. Paraprotein/FLC are also directly pathogenic in other plasma cell disorders, such as monoclonal gammopathy of renal significance (MGRS) or paraproteinemic-related neuropathies, such as monoclonal gammopathy of undetermined significance (MGUS)-related neuropathy.³⁻⁵

Over the past two decades, an improved understanding of MM biology has resulted in the development of more effective therapies, leading to a step-wise prolongation of median overall survival to current 8 years for many patients.⁶ However, therapeutic resistance is inevitable, eventually leading to death. The prognosis of AL patients remains dismal, with no FDA approved drugs; limited therapeutic options; and profound morbidity and disability from paraprotein/FLC-mediated organ damage. Thus, there is an urgent need for developing therapies for treating plasma cell disorders.

### SUMMARY

This disclosure relates to compositions and methods of treating plasma cell disorders, and/or disorders associated with protein secretion, production, or deposition, wherein the protein secretion, production, or deposition is pathogenic.

In one aspect, the disclosure relates to methods of treating a subject (e.g., a human) having a disorder associated with protein secretion, production, or deposition, that is pathogenic. The methods involve administering to the subject an effective amount of a composition comprising a Botulinum neurotoxin (BoNT) agent comprising a heavy chain and a light chain, wherein the BoNT inhibits the protein secretion, production, or deposition, that is pathogenic, thereby treating the disorder.

In some embodiments, the BoNT agent is a chimeric Botulinum neurotoxin. In some embodiments, the chimeric BoNT agent targets plasma cells. In some embodiments, the heavy chain of the chimeric BoNT agent targets one or more of markers selected from the group consisting of CD138, CD38, CD78, CD319, IL-6 receptor, and B-cell maturation antigen (BCMA). In some embodiments, the light chain of the chimeric BoNT agent cleaves soluble N-ethytmaleimide-sensitive factor attachment protein receptor (SNARE).

In some embodiments, the disorder is a plasma cell disorder.

In some embodiments, one or more plasma cells in the subject have an increased synthesis and/or secretion of paraprotein.

In some embodiments, one or more plasma cells in the subject have an increased synthesis and/or secretion of free light chains (FLC).

In some embodiments, the plasma disorder is multiple myeloma, Amyloid light-chain (AL) amyloidosis, monoclonal gammopathy of undermined significance (MGUS), monoclonal gammopathy of renal significance (MGRS), paraproteinimic neuropathy, polyneuropathy, organomegaly, endocrinopathy monoclonal gammopathy and skin changes syndrome (POEMS), non-AL amyloidosis, or a cancer whose pathogenic mechanism involves, or is due to, a secreted protein. In some embodiments, the cancer is an insulinoma, a gastrinoma, a secreting adrenal tumor, an adenoma, a parathyroid adenoma, a pituitary adenoma, a carcinoid tumor, an adenocarcinoma, a pancreatic cancer, a breast cancer, an ovarian cancer or a colon cancer.

In some embodiments, the subject has a tumor characterized by high protein secretion. In some embodiments, the tumor is an adenocarcinoma. The adenocarcinoma can be of the pancreas, breast, or colon.

In some embodiments, the subject is a human.

In some embodiments, the subject is not subjected to chemotherapy.

In some embodiments, the subject is also administered a proteasome inhibitor.

In one aspect, the disclosure also provides methods of treating a subject (e.g., a human) having a disorder associated with protein secretion, production, or deposition, that is pathogenic. The methods comprise administering to the subject an effective amount of a composition comprising a nucleic acid that encodes a BoNT light chain.

In some embodiments, the BoNT light chain is a Botulinum E light chain, or a mutant Botulinum E light chain. In certain instances, the mutant Botulinum E light chain comprises a K224D mutation (see, e.g., Chen and Barbieri, PNAS 106(23):9180-9184 (2009)). In some instances, the mutant Botulinum E light chain has 1 to 10 (e.g., 1, 2, 3, 4, 5, 6, 7, 8, 9, 10) amino acid substitutions relative to SEQ ID NO:5.

In some embodiments, the nucleic acid is delivered by a lentiviral vector.

In another aspect, the disclosure features methods of treating a subject (e.g., human) having a disorder associated with protein secretion, production, or deposition, that is pathogenic. The methods comprise administering to the subject an effective amount of a composition comprising a BoNT light chain.

In some embodiments, the BoNT light chain is a Botulinum E light chain, or a mutant Botulinum E light chain. In certain instances, the mutant Botulinum E light chain comprises a K224D mutation. In some instances, the mutant Botulinum E light chain has 1 to 10 (e.g., 1, 2, 3, 4, 5, 6, 7, 8, 9, 10) amino acid substitutions relative to SEQ ID NO:5.

In yet another aspect, the disclosure features a composition comprising a BoNT light chain and a proteasome inhibitor. In some instances, the BoNT light chain is a Botulinum E light chain, or a mutant Botulinum E light chain. In certain instances, the mutant Botulinum E light chain comprises a K224D mutation. In some instances, the mutant Botulinum E light chain has 1 to 10 (e.g., 1, 2, 3, 4, 5, 6, 7, 8, 9, 10) amino acid substitutions relative to SEQ ID NO:5. In some instances, the proteasome inhibitor is bortezomib, carfilzomib, ixazomib, salinosporamide A, NPI-0052, peptide boronate (MLN9708 or CEP-18770), or epoxyketone (ONX 0912). In some embodiments, the proteasome inhibitor is bortezomib, carfilzomib, ixazomib, marizomib (NPI-0052), peptide boronate (delanzomib), or epoxyketone (oprozimib). In certain instances, the composition is a pharmaceutical composition and comprises a pharmaceutically acceptable carrier.

In a further aspect, the disclosure features methods of treating a subject (e.g., human) having a disorder associated with protein secretion, production, or deposition, that is pathogenic. The methods comprise administering to the subject an effective amount of a composition comprising a BoNT light chain and a proteasome inhibitor. In some embodiments, the BoNT light chain is a Botulinum E light chain, or a mutant Botulinum E light chain. In certain instances, the mutant Botulinum E light chain comprises a K224D mutation. In some instances, the mutant Botulinum E light chain has 1 to 10 (e.g., 1, 2, 3, 4, 5, 6, 7, 8, 9, 10) amino acid substitutions relative to SEQ ID NO:5. In some instances, the proteasome inhibitor is bortezomib, carfilzomib, ixazomib, salinosporamide A (NPI-0052), peptide boronate (MLN9708 or CEP-18770), or epoxyketone (ONX 0912).

In some instances, in the above aspects, the disorder is a plasma cell disorder. In some instances, the plasma disorder is multiple myeloma, Amyloid light-chain (AL) amyloidosis, monoclonal gammopathy of undermined significance (MGUS), MGRS, paraproteinimic neuropathy, polyneuropathy, organomegaly, endocrinopathy monoclonal gammopathy and skin changes syndrome (POEMS), non-AL amyloidosis, or a cancer whose pathogenic mechanism involves, or is due to, a secreted protein. In some embodiments, the cancer is an insulinoma, a gastrinoma, a secreting adrenal tumor, an adenoma, a parathyroid adenoma, a pituitary adenoma, a carcinoid tumor, an adenocarcinoma, a pancreatic cancer, a breast cancer, an ovarian cancer or a colon cancer.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A. Immunofluorescence in 4 MM cell lines with increased sensitivity to PI (from left to right) shows baseline accumulation of polyUb proteins in sensitive but not resistant cell lines (top panels); treatment with bortezomib (btz) leads to increased fluorescence in all cell lines consistent with increased proteotoxicity (bottom panels).

FIG. 1B. Primary bone marrow MM cells from two MM patients (CD138+, right panels) show baseline accumulation of polyUb protein, overlapping with immunoglobulin light chain, consistent with baseline accumulation of misfolded FLC. Non-MM, bone marrow cells (CD138−, left panel) show absent baseline (top panel) and only modest polyUb proteins accumulation upon high dose btz treatment (lower panel).

FIG. 2A. Inhibition of de-novo protein synthesis via cycloheximide (CHX, 1 μg/mL) causes decreased bortezomib-induced apoptosis in MM.1S cells.

FIG. 2B. CHX decreases polyUb (bottom panels) in MM.1S cells untreated (left panels) or treated with btz (right panels).

FIG. 2C. Increased protein misfolding via ER stressor tunicamycin (Tm, 2.5 μg/mL) sensitizes U266 cells to Btz-induced apoptosis (Btz, 10 nM).

FIG. 3A. Western blot of whole cell lysate from ALMC1, ALMC2 and KMS11 cell lines showing abundant expression of IgG and λ light chain in ALMC1 and ALMC2. KMS11 synthesizes κ light chain only (previously reported as IgGK, production of light chain only was proven via western blot) and is shown as control. GAPDH is used as loading control.

FIG. 3B. 500,000 ALMC1 or ALMC2 cells were seeded for 4 hours. Supernatant was then harvested and 5 microL loaded and run into a western blot to assess secretion of IgG and λ light chain. Secreted λ light chain can be detected as monomer (lower duplex band) or a dimer (upper duplex band).

FIG. 4. Western blot of whole cell lysate from ALMC1 and ALMC2 show expression of SNAP23 and SYNTAXIN-4. GAPDH was used as loading control.

FIG. 5. Expression of Botulinum light chain E (LcE) and mutant light chain E (LcE*) in ALMC2 leads to loss of viability.

FIG. 6. Expression of Botulinum mutant light chain E results in cleavage of SNAP23, which is consistent with on target activity.

### DETAILED DESCRIPTION

Multiple myeloma (MM) and AL amyloidosis (AL) are diseases of clonal plasma cell (PC) proliferation and hyper-secretion of monoclonal immunoglobulin (paraprotein) and/or free light chain (FLC). MM is the second most frequent blood cancer in the western world, with a peak incidence in the 7th decade of life. AL is a rare, rapidly fatal disorder characterized by deposition of amyloidogenic FLC in target organs, leading to failure and eventually death. Despite the development of therapies such as proteasome inhibitors (PI), MM/AL are currently incurable.

The present disclosure shows that MM cells have baseline excess protein synthesis/misfolding in the face of limited proteasome-mediated degradation. Proteasome inhibitors exacerbate this imbalance, leading to proteotoxicity and apoptosis. Proteotoxicity similarly underlies PI sensitivity in AL. While PI are effective in treating MM/AL, resistance is inevitable, underscoring an important, unmet therapeutic need.

Botulinum neurotoxin (BoNT) can reduce paraprotein secretion in PC, thus BoNT can be used in treating MM/AL. Targeted inhibition of paraprotein/FLC secretion via a BoNT agent is a feasible and effective therapeutic strategy for treating plasma cell disorders and/or disorders associated with protein secretion, production, or deposition, wherein the protein secretion, production, or deposition is pathogenic. The BoNT agent leads to decreased protein secretion and direct cytotoxicity against cells via exacerbation of baseline proteotoxicity mediated by retained cytoplasmic immunoglobulin/free light chain.

Disorders Associated with Protein Secretion, Production, or Deposition

As used herein, the term “disorder associated with protein secretion, production, or deposition” refers to a disorder associated with protein secretion, production, or deposition, wherein the protein secretion, production, or deposition is pathogenic.

Disorders associated with protein secretion, production, or deposition, that is pathogenic, include, but are not limited to, plasma cell disorders (e.g., multiple myeloma (MM), and AL amyloidosis), non-AL amyloidosis, and certain cancers.

As used herein, the term “plasma cell disorder” refers to a group of diseases or disorders characterized by clonal plasma cell (PC) proliferation and hyper-secretion of paraproteins (e.g., monoclonal immunoglobulin and/or free light chain (FLC)). These plasma disorders can be relapsed and/or refractory, when they recur after a remission and/or when they do not respond to treatment, respectively.

As used herein, the term “non-AL amyloidosis” refers to an amyloidogenic disorder in which proteins other than immunoglobulin light chain are responsible for amyloidogenic deposition (transthyretin (TTR), serum amyloid A (SAA), etc.).

The cancers associated with protein secretion, production, or deposition, that is pathogenic, include cancers whose pathogenic mechanism is primarily due to a secreted protein (insulinoma; gastrinoma; secreting adrenal tumor/adenoma such as those producing steroid hormones, aldosteron or catecholamines; parathyroid adenoma; pituitary adenoma; carcinoid tumors) and/or cancers potentially have a therapeutic window in which cancer cells are characterized by high protein secretion such as adenocarcinoma, particularly pancreatic cancer, breast cancer, ovarian cancer and colon cancer. Similarly, benign conditions such as hyperfunctioning thyroid nodules or parathyroid adenoma can be amenable to the treatments as described in this disclosure.

Plasma cell disorders As used herein, the term “plasma cell disorders” refer to a group of diseases or disorders characterized by clonal plasma cell (PC) proliferation and hyper-secretion of paraproteins (e.g., monoclonal immunoglobulin and/or free light chain (FLC)).

Non-limiting examples of plasma cell disorders include monoclonal gammopathy of undermined significance (MGUS), multiple myeloma (MM), Waldenström macroglobulinemia (WM), light chain amyloidosis (AL), solitary plasmacytoma (e.g., solitary plasmacytoma of bone, or extramedullary plasmacytoma), polyneuropathy, organomegaly, endocrinopathy monoclonal gammopathy and skin changes syndrome (POEMS), and heavy-chain disease. MGUS, smoldering MM, and symptomatic MM represent a spectrum of the same disease. Other plasm cell disorders include, e.g., Monoclonal Gammopathy of Renal Significance (MGRS), MGUS-associated neuropathy, and other paraproteinemic neuropathy.

Symptomatic or active multiple myeloma is characterized by more than 10% BM infiltration by clonal plasma cells and/or biopsy proven plasmacytoma in addition to any level of monoclonal protein and the presence of end-organ damage that consists of a myeloma defyning event in the form of any of the CRAB criteria (hypercalcemia, renal insufficiency, anemia, or bone lesions which are deemed related to the plasma cell clone) or any of the new biomarker of malignancy (BM involvement by equal or greater than 60% clonal plasma cell; a ratio of involved versus uninvolved FLC equal or exceeding 100; and/or the presence of more than one bone lesion on MRI (Kyle R. A. et al., Leukemia, 23:3-9 (2009); Rajkumar V. S. et al, Lancet Oncology, 15:12, 2014). MM is a plasma cell malignancy that characteristically involves extensive infiltration of bone marrow (BM), and occasionally the formation of plasmacytoma, as discrete clusters of malignant plasma cells inside or outside of the BM space (Kyle R. A. et al., N. Engl. J. Med., 351:1860-73 (2004)). Consequences of this disease are numerous and involve multiple organ systems. Disruption of BM and normal plasma cell function leads to anemia, leukopenia, hypogammaglobulinemia, and thrombocytopenia, which variously result in fatigue, increased susceptibility to infection, and, less commonly, increased tendency to bleed. Disease involvement in bone creates osteolytic lesions, produces bone pain, and may be associated with hypercalcemia (Kyle R. A. et al., Blood, 111:2962-72 (2008)).

AL amyloidosis is a rare rapidly fatal disorder characterized by deposition of amyloidogenic FLC in target organs, leading to failure and eventually death. Diagnosis of AL amyloidosis is typically delayed due to the insidious nature of clinical presentation, leading to recognition often in advanced stages which negatively affects outcome. The diagnosis of AL amyoidosis requires biopsy proven demonstration of amyloid deposition in any tissue via Congo red stain and identification of light chain as the amyloidogenic protein via mass spectrometry or immunoelectromycroscopy; presence of amyloid-related organ damage or syndrome; and identification of a monoclonal gammopathy based on presence of M spike and/or sFLC and presence of BM infiltration by clonal plasma cells. Amyloidogenic protein causes the pathognomonic “apple-green” pattern of polarized light refringence upon Congo red staining. The pattern of organ involvement by AL amyloid influences the clinical presentation of AL amyloidosis. For instance, cardiac involvement presents with heart failure secondary to restrictive or dilated cardiomyopathy; kidney involvement presents with nephrotic syndrome; liver involvement results in hepatic failure; gastrointestinal tract involvement manifests as diarrhea or gastrointestinal bleed; nervous system involvement typically presents as distal, sensory peripheral neuropathy; while soft tissue involvement results in periorbital purpura and easy bruisibility. AL amyloidosis is a true, distinct clinical entity from MM and only a minority of patients presents with an overlaps syndrome where diagnostic criteria for both AL and MM are met (Gertz et al; Am J of Hematology, 2016). MGUS is characterized by a serum monoclonal protein, <30 g/L, <10% plasma cells in the bone marrow, and absence of end-organ damage (Kyle R. A. et al., Leukemia, 23:3-9 (2009)). Recent studies suggest that an asymptomatic MGUS stage consistently precedes multiple myeloma (MM) (Landgren O. et al., Blood, 113:5412-7 (2009)). MGUS is present in 3% of persons >50 years and in 5% >70 years of age. The risk of progression to MM or a related disorder is 1% per year (Kyle R. A. et al., Clin. Lymphoma Myeloma, 6:102-14 (2005)). Patients with risk factors consisting of an abnormal serum free light chain ratio, non-immunoglobulin G (IgG) MGUS, and an elevated serum M protein >/=15 g/l had a risk of progression at 20 years of 58%, compared with 37% among patients with two risk factors, 21% for those with one risk factor, and 5% for individuals with no risk factors (Rajkumar S. V., Br. J. Haematol., 127:308-10 (2004)). The cumulative probability of progression to active MM or amyloidosis was 51% at 5 years, 66% at 10 years and 73% at 15 years; the median time to progression was 4.8 years (Rajkumar S. V., Blood Rev., 21:255-65, (2007)).

SMM is characterized by having a serum immunoglobulin (Ig) G or IgA monoclonal protein of 30 g/L or higher and/or 10% or more plasma cells in the bone marrow but no evidence of end-organ damage or malignancy-defining biomarkers (Rajkumar et al, Lancet, 2014). A study of the natural history of SMM suggests that there are 2 different types: evolving smoldering MM and non-evolving Smoldering MM (Dimopoulos M. et al., Leukemia, 23(9):1545-56 (2009)). Evolving SMM is characterized by a progressive increase in M protein and a shorter median time to progression (TTP) to active multiple myeloma of 1.3 years. Non-evolving SMM has a more stable M protein that may then change abruptly at the time of progression to active multiple myeloma, with a median TTP of 3.9 years.

Waldenstrom's macrogloubulinemia (WM), termed lymphoplasmacytic lymphoma in the World Health Organization classification, is an indolent lymphoid malignancy composed of mature plasmacytoid lymphocytes that produce monoclonal IgM (Leleu X. et al., Cancer Lett., 270: 95-107 (2008)). The disease affects predominantly older patients, who present with anemia, lymphadenopathy, purpura, splenomegaly, elevated serum viscosity, neurologic signs and symptoms, or combinations of these findings. Lytic bone lesions are typically absent. The lymphoma cells may express a variety of markers, including CDS, CD19, CD20, CD38, and surface or cytoplasmic Ig. Symptoms may be due to tumor infiltration (marrow, spleen, or lymph nodes), circulating IgM macroglobulin (hyperviscosity, cryoglobulinemia, or cold agglutinin hemolytic anemia), and tissue deposition of IgM or other proteins (neuropathy, glomerular disease, and/or amyloid).

Paraprotein/FLC have been recognized as directly pathogenic in a number of patients with plasma cell disorders not meeting criteria for MM/AL, but presenting with symptoms/signs such as proteinuria, renal failure or neuropathy which are direct consequence of paraprotein/FLC toxicity. While not per se fatal, these conditions can significantly affect quality of life and result in major disability such as end stage renal disease requiring renal replacement therapy or limb plegia.

MGRS and paraproteinemic neuropathy are recently identified clinical entities where a standard therapeutic approach has not yet been identified. Although the plasma cell clone is not directly pathogenic in these conditions, therapies are directed at killing the plasma cell clone so as to halt the FLC production. Similarly, MGRS and other paraprotein-related non-cancerous conditions can be treated with chemotherapy, although the plasma cell clone per se is not directly pathogenic.

Botulinum neurotoxin agent As used herein, the term “Botulinum neurotoxin agent” or “BoNT agent” refers to an agent comprising Botulinum toxin, chimeric Botulinum toxin, engineered Botulinum toxin, or a protein or peptide derived from Botulinum toxin. In some embodiments, a nucleic acid encoding Botulinum toxin, chimeric Botulinum toxin, engineered Botulinum toxin, a protein or peptide derived from Botulinum toxin can be administered to a subject in need thereof. Botulinum neurotoxin (BoNT) is a protein produced by the genus Clostridium of

Gram positive bacteria. More than 40 different serotypes of BoNT exist in nature. The mature BoNT is composed of a light (L) and a heavy (H) chain which are linked via a single disulfide bond and a linker peptide. The C terminus of the H chain binds to pre-synaptic axon of neuromuscular junctions and facilitates endocytosis of the BoNT. The N terminus of the H chain mediates the cytosolic translocation of the L chain from the endocytic vesicle. The L chain encodes the catalytic activity of the neurotoxin, a metalloprotease with specific activity against certain SNARE proteins. The overall effect of the BoNT is inhibition of acetylcholine release from the presynaptic axon, resulting in flaccid paralysis. (Rossetto O et al, Nature Reviews Microbiology, 12 353:549, 2014).

The sequences for Botulinum neurotoxin are shown below:

Botulinum D LC sequence (SEQ ID NO: 1):
YYDPSYLSTDEQKDTFLKGIIKLFKRINERDIGKKLINYLVVGSPFMGDSSTPEDTFDFT
RHTTNIAVEK
FENGSWKVTNIITPSVLIFGPLPNILDYTASLTLQGQQSNPSFEGFGTLSILKVAPEFLL
TFSDVTSNQS
SAVLGKSIFCMDPVIALMHELTHSLHQLYGINIPSDKRIRPQVSEGFFSQDGPNVQFEEL
YTFGGLDVEI
IPQIERSQLREKALGHYKDIAKRLNNINKTIPSSWISNIDKYKKIFSEKYNFDKDNTGNF
VVNIDKFNSL
YSDLTNVMSEVVYSSQYNVKNRTHYFSRHYLPVFANILDDNIYTIRDGFNLTNKGFIENS
GQNIERNPA
LQKLSSESVVDLFTKVCLRLTKNS
GHRH amino acid 1-40 (SEQ ID NO: 3):
MPLWVFFFVILTLSNSSHCSPPPPLTLRMRRYADAIFTNS
Botulinum D HC sequence (SEQ ID NO: 2)
WPVKDFNYSDPVNDNDILYLRIPQNKLITTPVKAFMITQNIWVIPERFSSDTNPSLSKPP
RPTSKYQS
YYDPSYLSTDEQKDTFLKGIIKLEKRINERDIGKKLINYLVVGSPFMGDSSTPEDTEDFT
RHTTNIAVEK
FENGSWKVINIITPSVLIFGPLPNILDYTASLTLQGQQSNPSFEGEGILSILKVAPEFLL
TESDVISNQS
SAVLGKSIFCMDPVIALMHELTHSLHQLYGINIPSDKRIRPQVSEGFFSQDGPNVQFEEL
YTEGGLDVEI
IPQIERSQLREKALGHYKDIAKRLNNINKTIPSSWISNIDKYKKIFSEKYNEDKDNIGNE
VVNIDKENSL
YSDLINVMSEVVYSSQYNVKNRTHYFSRHYLPVFANILDDNIYTIRDGENLINKGENIEN
SGQNIERNPA
LQKLSSESVVDLFTKVCLRLTKNS
RDDSTCIKVKNNRLPYVADKDSISQEIFENKIITDETNVQNYSDKF
SLDESILDGQVPINPEIVDPLLPNVNMEPLNLPGEEIVEYDDITKYVDYLNSYYYLESQK
LSNNVENITL
TTSVEEALGYSNKIYTELPSLAEKVNKGVQAGLELNWANEVVEDFTTNIMKKDTLDKISD
VSVIIPYIGP
ALNIGNSALRGNENQAFATAGVAELLEGFPEFTIPALGVETFYSSIQEREKIIKTIENCL
EQRVKRWKDS
YQWMVSNWLSRITTQFNHINYQMYDSLSYQADAIKAKIDLEYKKYSGSDKENIKSQVENL
KNSLDVKISE
AMNNINKFIRECSVTYLEKNMLPKVIDELNKFDLRIKTELINLIDSHNIILVGEVDRLKA
KVNESFENTM
PFNIFSYTNNSLLKDIINEYENSINDSKILSLQNKKNALVDTSGYNAEVRVGDNVQLNTI
YINDFKLSSS
GDKIIVNLNNNILYSAIYENSSVSFWIKISKDLINSHNEYTIINSIEQNSGWKLCIRNGN
IEWILQDVNR
KYKSLIFDYSESLSHIGYINKWFFVTITNNIMGYMKLYINGELKQSQKIEDLDEVKLDKT
IVEGIDENID
ENQMLWIRDENIFSKELSNEDINIVYEGQILRNVIKDYWGNPLKEDTEYYIINDNYIDRY
IAPESNVLVL
VQYPDRSKLYTGNPITIKSVSDKNPYSRILNGDNIILHMLYNSRKYMIIRDTDTIYATQG
GECSQNCVYA
LKLQSNLGNYGIGIFSIKNIVSKNKYCSQIESSFRENTMLLADIYKPWRFSEKNAYTPVA
VTNYETKLLS
TSSFWKFISRDPGWVE
Botulinum B LC sequence (SEQ ID NO: 4)
PVTINNFNYNDPIDNNNIIMMEPPFARGTGRYYKAFKITDRIWIIPERYTFGYKPEDFNK
SSGIFNRDVCEYYDPDYLNTNDKKNIFLQTMIKLFNRIKSKPLGEKLLEMIINGIPYLGD
RRVPLEEFNTNIASVIVNKLISNPGEVERKKGIFANLIIFGPGPVLNENETIDIGIQNHF
ASREGFGGIMQMKFCPEYVSVFNNVQENKGASIFNRRGYFSDPALILMHELIHVLHGLYG
IKVDDLPIVPNEKKFFMQSTDAIQAEELYTFGGQDPSIITPSTDKSIYDKVLQNFRGIVD
RLNKVLVCISDPNININIYKNKFKDKYKFVEDSEGKYSIDVESFDKLYKSLMFGFTETNI
AENYKIKTRASYFSDSLPPVKIKNLLDNEIYTIEEGFNISDKDMEKEYRGQNKAINKQAY
EEISKEHLAVYKIQMCKSVK
Botulinum E LC sequence (SEQ ID NO: 5)
PKINSFNYNDPVNDRTILYIKPGGCQEFYKSFNIMKNIWIIPERNVIGTTPQDFHPPTSL
KNGDSSYYDPNYLQSDEEKDRFLKIVTKIFNRINNNLSGGILLEELSKANPYLGNDNTPD
NQFHIGDASAVEIKFSNGSQDILLPNVIIMGAEPDLFETNSSNISLRNNYMPSNHRFGSI
AIVTFSPEYSFRFNDNCMNEFIQDPALTLMHELIHSLHGLYGAKGITTKYTITQKQNPLI
TNIRGINIEEFLIFGGIDLNIITSAQSNDIYTNLLADYKKIASKLSKVQVSNPLLNPYKD
VFEAKYGLDKDASGIYSVNINKFNDIFKKLYSFTEFDLRIKFQVKCRQTYIGQYKYFKLS
NLLNDSIYNISEGYNINNLKVNFRGQNANLNPRIITPITGRGLVKKIIRFCKNIVSVKGI
R
An Exemplary Mutant Botulinum E LC sequence (E LC sequence
with K224D mutation) (SEQ ID NO: 6)
PKINSFNYNDPVNDRTILYIKPGGCQEFYKSFNIMKNIWIIPERNVIGTTPQDFHPPTSL
KNGDSSYYDPNYLQSDEEKDRFLKIVTKIFNRINNNLSGGILLEELSKANPYLGNDNTPD
NQFHIGDASAVEIKFSNGSQDILLPNVIIMGAEPDLFETNSSNISLRNNYMPSNHRFGSI
AIVTFSPEYSFRFNDNCMNEFIQDPALTLMHELIHSLHGLYGADGITTKYTITQKQNPLI
TNIRGINIEEFLIFGGIDLNIITSAQSNDIYTNLLADYKKIASKLSKVQVSNPLLNPYKD
VFEAKYGLDKDASGIYSVNINKFNDIFKKLYSFTEFDLRIKFQVKCRQTYIGQYKYFKLS
NLLNDSIYNISEGYNINNLKVNFRGQNANLNPRIITPITGRGLVKKIIRFCKNIVSVKGI
R
Botulinum E HC sequence (SEQ ID NO: 7)
KSICIEINNGELFFVASENSYNDDNINTPKEIDDIVISNNNYENDLDQVILNFNSESAPG
LSDEKLNLTIQNDAYIPKYDSNGTSDIEQHDVNELNVFFYLDAQKVPEGENNVNLTSSID
TALLEQPKIYTFFSSEFINNVNKPVQAALFVSWIQQVLVDFTTEANQKSTVDKIADISIV
VPYIGLALNIGNEAQKGNFKDALELLGAGILLEFEPELLIPTILVFTIKSFLGSSDNKNK
VIKAINNALKERDEKWKEVYSFIVSNWMTKINTQFNKRKEQMYQALQNQVNAIKTIIESK
YNSYTLEEKNELTNKYDIKQIENELNQKVSIAMNNIDRFLTESSISYLMKIINEVKINKL
REYDENVKTYLLNYIIQHGSILGESQQELNSMVIDTLNNSIPFKLSSYTDDKILISYFNK
FFKRIKSSSVLNMRYKNDKYVDTSGYDSNININGDVYKYPTNKNQFGIYNDKLSEVNISQ
NDYIIYDNKYKNFSISFWVRIPNYDNKIVNVNNEYTIINCMRDNNSGWKVSLNHNEIIWT
FEDNRGINQKLAFNYGNANGISDYINKWIFVTITNDRLGDSKLYINGNLIDQKSILNLGN
IHVSDNILFKIVNCSYTRYIGIRYFNIFDKELDETEIQTLYSNEPNTNILKDFWGNYLLY
DKEYYLLNVLKPNNFIDRRKDSTLSINNIRSTILLANRLYSGIKVKIQRVNNSSTNDNLV
RKNDQVYINFVASKTHLFPLYADTATTNKEKTIKISSSGNRFNQVVVMNSVGNCTMNFKN
NNGNNIGLLGFKADTVVASTWYYTHMRDHINSNGCFWNFISEEHGWQEK

For cells associated with paraprotein hypersecretion (e.g., MM cells), these cells usually have baseline excess protein synthesis/misfolding in the face of limited proteasome-mediated degradation. BoNT can further reduce protein secretion in these cells, leading to proteotoxicity and apoptosis. As BoNT can inhibit the processes of lysosome or autophagosome formation, BoNT can be clinically useful in treating plasma cell disorders (e.g., MM/AL) or disorders associated with protein secretion, production, or deposition by exacerbating proteotoxicity. In fact, inhibition of autophagy can be used as a therapeutic approach to increase sensitivity to PI and/or overcome clinical resistance, as autophagy/aggresome are upregulated in cells treated with PI.

Furthermore, the BoNT domains can be engineered to target a specific cell population (H chain engineering) and/or a specific SNARE protein (L chain), resulting in targeted inhibition of protein secretion. Targeted inhibition of protein secretion via BoNT agent is a more effective therapeutic strategy in plasma cell disorders and/or disorders associated with protein secretion, production, or deposition. In the case of MM/AL and/or other plasma cell disorders characterized by paraprotein/FLC-mediated damage, the targeted inhibition leads to inhibition of paraprotein/FLC secretion and direct cytotoxicity against MM/AL cells via exacerbation of baseline proteotoxicity.

The heavy chain of BoNT can be engineered to target cell surface markers such as, but not limited to, CD138, CD38, CD78, CD319, IL-6 receptor, and B-cell maturation antigen (BCMA). In some embodiments, the heavy chain domain can target plasma cells. In some embodiments, the heavy chain can be linked to an antibody or antibody fragment thereof, wherein the antibody or antibody fragment thereof binds to a plasma cell (e.g., through binding markers such as, but not limited to, CD138, CD38, CD78, CD319, IL-6 receptor, and BCMA.

In some embodiments, the heavy chain of BoNT can comprise an antibody, or an antigen binding fragment thereof, e.g., Fab, a scFv (single-chain variable fragments), a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain.

The light chain of BoNT can be engineered to cleave SNARE proteins. There are several different types of SNARE proteins, e.g., t- and v-SNAREs, syntaxin-4, SNAP23, SNAP25 and VAMP-2 etc. Some of these SNARE proteins are responsible for immunoglobulin secretion in plasma cells. There are different serotypes of BoNT. Each serotype has different specificity for specific SNARE proteins. The light chain of an appropriate serotype can be selected for targeting SNARE of interest. In some embodiments, the light chain of BoNT can also be engineered to target specific SNARE, e.g., t- and v-SNAREs, syntaxin-4, SNAP23, SNAP25 and/or VAMP-2. The target sites of BoNT are shown in the table below, and are described, e.g., Zhang, Sicai, et al. “Identification and characterization of a novel botulinum neurotoxin.” Nature communications 8 (2017): 14130; and Lebeda, Frank J., et al. “The zinc-dependent protease activity of the botulinum neurotoxins.” Toxins 2.5 (2010): 978-997, both of which are incorporated by reference in its entirety.

TABLE 1

| | | | Substrate |
|---|---|---|---|
| Neurotoxin | Target Substrate | Cleavage Site | Localization |
| BoNT A | SNAP-25 | Glnl97-Arg198 | presynaptic plasma |
| | | | membrane |
| BoNTB | VAMP | Gln76-Phe77 | synaptic vesicle |
| BoNTC1 | SNAP-25 | Arg198-Ala199 | presynaptic plasma |
| | Syntaxin 1a | Lys253-Ala254 | membrane |
| | Syntaxin 1b | Lys252-Ala253 | |
| BoNT E | SNAP-25 | Arg180-IIe 181 | presynaptic plasma |
| | | | membrane |
| BoNT F | VAMP1 | Gln60-Lys61 | synaptic vesicle |
| | VAMP2 | Gln58-Lys59 | |
| BoNT G | VAMP1 | Ala83-Ala84 | synaptic vesicle |
| | VAMP2 | Ala81-Ala82 | |
| BoNT X | VAMP1 | | synaptic vesicle |
| | VAMP2 | | |
| | VAMP3 | | |

Thus, the BoNT agent can be used to treat plasma cell disorders and other diseases where protein production/deposition is directly pathogenic, such as amyloidosis. The chimeric BoNT H chain can be engineered to recognize target cells (e.g., plasma cells), while the L chain can be engineered to cleave specific SNARE proteins responsible for the secretion of the target protein (i.e. paraprotein/free light chain), resulting in specific inhibition of pathogenic protein secretion and induction of cytotoxicity.

In some embodiments, the pathogenic proteins in patients affected by these disorders can be identified, e.g., by sequencing or PCR-based sequencing. The BoNT can be further engineered to recognizing the pathogenic protein epitope to maximize specificity against target cells.

In some embodiments, the BoNT heavy chain is a serotype A, serotype B, serotype C, serotype D, serotype E, serotype F, or serotype X heavy chain. In some embodiments, the BoNT heavy chain is a serotype D heavy chain. In some embodiments, the BoNT heavy chain comprises a sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% identical to the BoNT heavy chain sequence as described herein. The heavy chain is responsible for cell specificity. The heavy chain can be engineered to target a cell type of interest. For example, heavy chains that target plasma cells would have to be necessarily different than those used to specifically target other disorders (e.g., insulinoma, a gastrinoma, a secreting adrenal tumor, an adenoma, a parathyroid adenoma, a pituitary adenoma, a carcinoid tumor, an adenocarcinoma, a pancreatic cancer, a breast cancer, an ovarian cancer or a colon cancer).

In some embodiments, the BoNT light chain is a serotype A, serotype B, serotype C, serotype D, serotype E, serotype F, or serotype X light chain. In some embodiments, the BoNT light chain is a serotype E light chain or mutant serotype E light chain (e.g., comprising K224D mutation). In some embodiments, the BoNT light chain comprises a sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% identical to the BoNT light chain sequence as described herein. In some instances, the BoNT light chain has the amino acid sequence set forth in SEQ ID NO:5 or 6 except having 1 to 10 (e.g., 1, 2, 3, 4, 5, 6, 7, 8, 9, 10) amino acid substitutions relative to SEQ ID NO:5 or 6.

BoNT are reviewed in Lebeda, Toxins, 2:978-997 (2010) and also described in Zhang et al., Nat Commun., DOI: 10.1038/ncomms14130 and Barbieri et al., PNAS 106(23):9180-9184, and the botulinum neurotoxin resource, BotDB (http://botdb.abcc.ncifcrf.gov). These materials are all incorporated by reference herein in their entireties.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

### Methods of Treatment

The methods described herein include methods for the treatment of a subject having plasma cell disorders and/or disorders associated with protein secretion, production, or deposition. In these subjects, the methods described herein can directly inhibit the secretion of the pathogenic protein. Inhibition of pathogenic protein (e.g., paraprotein, FLC) secretion will lead to overwhelming proteotoxic stress, resulting in apoptosis of pathogenic cells. As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

Generally, the methods include administering a therapeutically effective amount of a composition comprising or consisting of Botulinum toxin agents as described herein, to a subject who is in need of, or who has been determined to be in need of, such treatment.

As used in this context, to “treat” means to ameliorate at least one symptom of the disorder. Often, the treatment can result in slowing or stopping the progression of the disorder, and in some cases, can reverse the progression of the disorder and/or cure the disorder. In some embodiments, the treatment results in the reduction of pathogenic protein secretion, inhibition of the pathogenic cell activity, and/or the death of the pathogenic cell.

In some embodiments, the agent can be one or more nucleic acids that encode a BoNT light chain and/or BoNT heavy chain. In some embodiments, the nucleic acid encodes a BoNT light chain. In some embodiments, the BoNT light chain is a BoNT serotype E or mutant serotype E light chain.

In some embodiments, the BoNT agent can be used in combination with some other therapeutic agents, e.g., chemotherapy agents, proteasome inhibitors, HDAC 6 inhibitors, soluble N-ethytmaleimide-sensitive factor attachment protein receptor (SNARE) inhibitor (e.g., SNARE siRNA), tetanus toxin, endoplasmic reticulum (ER) stressors, spiegelmer targeting immunoglobulins and/or FLC and NEOD001. Expression of tetanus toxin light chain in these pathogenic cells (e.g., MM cells) can result in cleavage of VAMP-2, increased intracellular retention of antibodies, and partial suppression of antibody secretion. NEOD001 is a monoclonal antibody binding misfolded FLC that has promising results in clinical trials in AL. These additional agents can be administered to a subject prior to, during, or after the administration of the BoNT agent to the subject.

In some embodiments, the BoNT agent is administered to a subject in need thereof who is not administered chemotherapy.

In fact, there is evidence supporting a protective role for autophagy in healthy tissues (such as cardiac myocytes) exposed to PI toxicity, raising concern that combination treatment of PI and autophagy inhibitors may prove to be clinically intolerable. Thus, the combination therapy with PI and a BoNT agent, or the combination therapy with an autophagy inhibitor and a BoNT agent, which targets SNAREs mediating autophagosome formation in a tissue specific manner, can represent a better tolerated and more efficacious treatment strategy.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic agent (i.e., an effective dosage) depends on the therapeutic agents selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic agents described herein can include a single treatment or a series of treatments.

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents which exhibit high therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from cell culture assays and animal studies can be used in formulating a range of dosage for use in humans. The dosage of such agents lies preferably within a range of circulating concentrations that include the ED50 with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC50 (i.e., the concentration of the test agent which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions and Methods of Administration

The methods described herein include the use of pharmaceutical compositions comprising or consisting of a BoNT agent as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous; oral, e.g., by mouth; inhalation; transdermal (e.g: via patch); transmucosal; and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active agent can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798, which is incorporated by reference in its entirety.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems. Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus (e.g., Adeno-Associated Virus Serotype 8 (AAV8) or Serotype 9 (AAV9)), pox virus or alphavirus. The recombinant vectors capable of expressing the nucleic acids of the invention can be delivered as described herein, and persist in target cells (e.g., stable transformants).

The pharmaceutical compositions can be included in a container, pack, or dispenser together with instructions for administration.

### EXAMPLES

The invention is further described in the following examples, which do not limit the scope of the invention described in the claims.

### Example 1: Proteotoxic Stress can Induce Cell Apoptosis

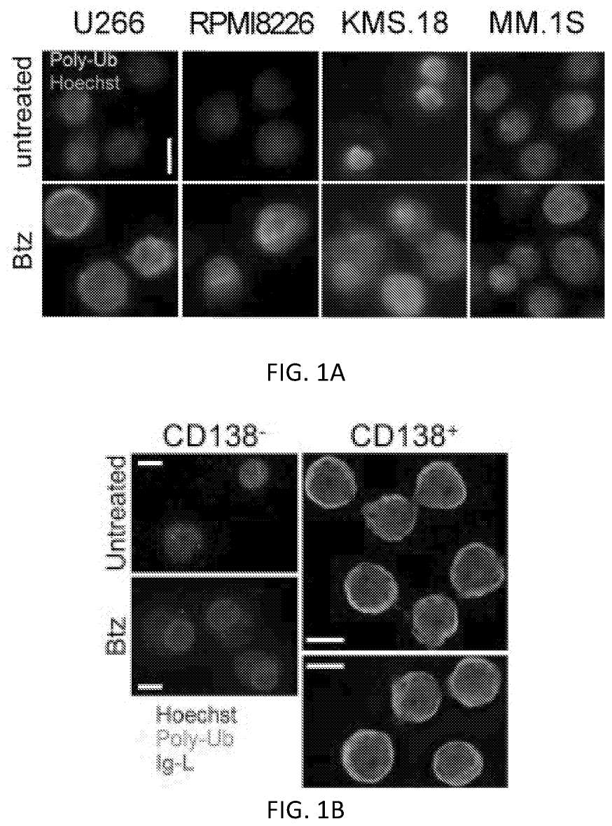

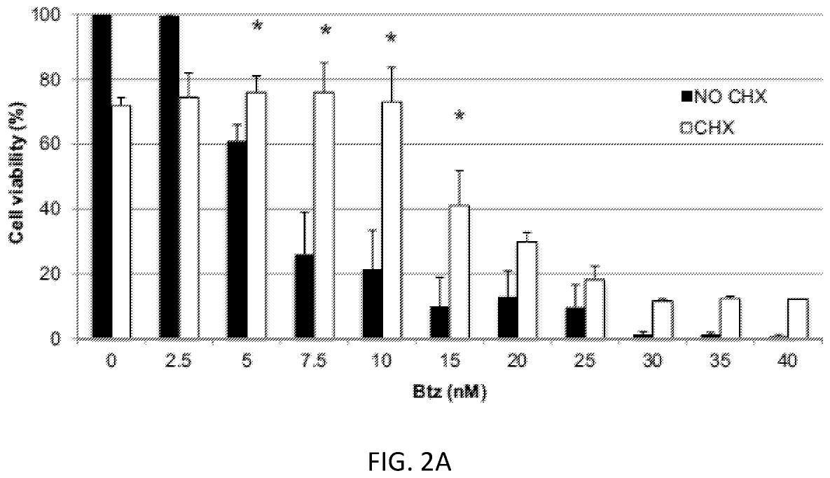

Proteasome inhibitors (PI) are small molecule inhibitors of the proteasome, a large, multicatalytic protease responsible for the degradation of most misfolded/aged polyubiquitinated (polyUb) proteins in eukaryotic cells. MM cells with baseline excess polyUb proteins and/or decreased proteasome activity are intrinsically sensitive to PI (FIGS. 1A and 1B). PI exacerbate this imbalance, leading to overwhelming proteotoxicity and apoptosis. Furthermore, decreased protein synthesis results in increased resistance to PI-induced apoptosis in MM, while increased protein misfolding strongly synergized with PI (FIGS. 2A-2C).

### Example 2: Chimeric BoNT Targeting Paraprotein/FLC in MM/AL

This example focuses on designing and optimizing chimeric BoNT specifically targeting paraprotein/FLC in MM/AL.

The experiments are designed to identify the optimal light chain (LC) serotype. Lentiviral vectors are used for LC expression. A panel of MM cell lines are transduced with lentivirus coding for a specific LC serotype or the backbone vector without insert (control). GFP is used as selection marker via fluorescent-activated sorting (FACS). Alternatively, an antibiotics can be used as selection markers. Viability (WST assay), apoptosis (annexin V/PI staining and flow cytometry), and paraprotein/FLC secretion (ELISA and western blot (WB) of supernatant) are assessed.

Protein lysates are obtained from transduced cells as a positive control to confirm cleavage of the LC-targeted SNARE. These are routinely used techniques in the lab. More than 6 authenticated MM cell lines are used. Their karyotype, FISH abnormalities and paraprotein isotype (IgG, IgA, IgE, etc.) and light chain (κ or λ) are well established. Synthesis and secretion of published paraprotein is confirmed in each cell line prior to the experiments.

The AL cell lines ALMC-1 and ALMC-2 are also used in the experiments. LC serotypes are scored based on the ability to decrease viability, induce apoptosis, and inhibit parparotein/FLC secretion.

At least one or more serotypes can be identified as cytotoxic for MM/AL cell lines. These will be selected for therapeutic use.

Optimal LC serotype can also be selected based on maximal inhibition of paraprotein/FLC secretion in most cell lines tested. These screening experiments will also provide data regarding whether different paraprotein isotypes (IgG, IgA, IgE, etc.), light chain (κ or λ), and amyloidogenic versus non-amyloidogenic FLC, have distinct SNARE requirements for secretion.

If cleavage of more than one SNARE is needed to significantly abate paraprotein/FLC secretion, BoNT can be engineered to simultaneously target multiple SNAREs.

Once an optimal LC serotype has been identified, intracellular retention of FLC/paraprotein via WB (with loading on a per cell, rather than per protein base) and IF is assessed.

A chimeric BoNT linking the previously identified optimal LC serotype to a heavy chain (HC) domain recognizing a specific receptor expressed universally by MM/AL cells can be created. The surface proteins CD138, CD38, and BCMA are all candidate targets for specific recognition of MM/AL cells.

### Example 3: In Vitro Validation of Chimeric BoNT Activity in Affecting Viability and Reducing Paraprotein/FLC Secretion

The chimeric BoNT can be validated in MM/AL cell lines. Dose and time course experiments can be performed in a panel of MM/AL cell lines to assess for decreased viability, apoptosis induction, and decreased secretion of paraprotein/FLC upon exposure to BoNT or control BoNT devoid of LC. Cell lysates are harvested after treatment with chimeric BoNT and are used to assess for cleavage of target SNAREs, confirming on target effect.

Chimeric BoNT against primary MM/AL cells isolated from patients are also tested. Briefly, newly diagnosed and/or relapsed and refractory MM/AL patients will be consented under IRB approved protocol prior to undergoing bone marrow aspirate and biopsy for diagnostic purposes. A heparinized sample of fresh bone marrow aspirate will be obtained during the procedure and will be processed the same day.

Bone marrow plasma is aliquoted and stored at −80° C. The remainder of the sample are diluted two folds with PBS or HBSS and then subjected to Ficoll-Paque PLUS (density 1.077±0.001 g/ml, GE Healthcare) density separation per protocol. Bone marrow mononuclear cells (BMMC) are carefully collected and washed once with PBS before undergoing red blood cell lysis (Boston Bioproducts, Ashland, Mass.). Following red cell lysis, BMMC will be washed once in PBS and once in MACS buffer before undergoing CD138+ magnetic bead positive selection (Miltenyi biosciences, Cambridge, Mass.).

CD138+ cells are washed twice in PBS before resuspension in RPMI 20% FBS medium and immediate use in dose-course experiments with chimeric BoNT. After 24-48 hours, supernatants of cells treated with increasing doses of chimeric BoNT or control (BoNT devoid of LC) will be collected and used in ELISA assay to detect FLC/paraprotein secretion. Cells are harvested for annexin V/PI apoptosis assay and WB analysis of SNARE cleavage if in sufficient amount.

As a control for specificity of BoNT against MM/AL cells, CD138− cells (negative fraction upon CD138+ magnetic bead selection) are also resuspended in RPMI 20% FBS and immediately seeded and treated with increasing doses of chimeric BoNT or control BoNT devoid of LC. Cells are harvested after 24-48 hours for annexin V/PI apoptosis assay and WB analysis of SNARE cleavage. It is expected that there is no induction of cytotoxicity and no SNARE cleavage by BoNT in these CD138− cells.

### Example 4: In Vivo Validation of Chimeric BoNT Activity in Affecting Viability and Reducing Paraprotein/FLC Secretion

In vivo validation of chimeric BoNT is evaluated in a mouse model routinely used in the lab. This is a humanized, plasmacytoma mouse model, in which a 1:1 mix of human MM cells and matrigel is injected subcutaneously (in either one or both flanks) of female, SCID beige mice. Over 2-3 week time, a palpable plasmacytoma develops, allowing longitudinal, volumetric assessment of tumor growth. This model can be used with a representative MM and a representative AL cell line. 14 mice per experiment are inoculated. Once all plasmacytoma have reached at least 5 mm diameter, the mice are divided into 2 cohorts of 7 mice each, distributed equally according to tumor volume. The control cohort receives a BoNT devoid of LC, and the experimental cohort receives the intact BoNT with both HC and LC. Tumor volume and weight are measured twice a week until protocol endpoints are met. Serum samples are also obtained twice weekly with serial tail vein/retro-orbital sampling to assess for paraprotein/FLC concentration via ELISA. Concentrations are normalized to tumor volume to estimate paraprotein/FLC secretion/cell.

The experiments are repeated twice for a total of 14 mice per cohort. These numbers provide at least 80% power to detect large differences in mean paraprotein/FLC secretion between control and experimental mice, with a one-sided t-test a error of 0.05 and difference in means equivalent to one standard deviation. The number of mice may need to be increased to detect a difference in mean tumor volume, assuming the effect of BoNT on tumor growth/survival may be less pronounced than on paraprotein/FLC secretion.

This mouse model has been routinely used in the lab for preclinical validation of investigational agent and can be used for assessment of anti-secretive and/or antiproliferative activity of BoNT against human MM/AL cell lines.

### Example 5: BoNT Activity Affects Cell Viability

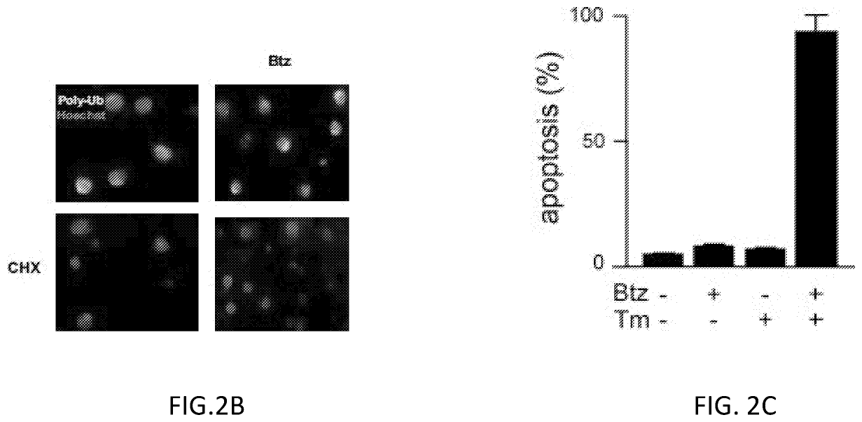

Western blot was performed for whole cell lysate from ALMC1, ALMC2 and KMS11 cell lines. The results showed that ALMC1 and ALMC2 cells express a large amount of IgG and λ light chain. KMS11 synthesized κ light chain only (previously reported as IgGK, production of light chain only was proven via western blot) and was shown as control. GAPDH was used as loading control (FIG. 3A). 500,000 ALMC1 or ALMC2 cells were then seeded for 4 hours. Supernatant was then harvested and 5 microL loaded and run into a western blot to assess secretion of IgG and λ light chain. Secreted lambda can be detected as monomer (lower duplex band) or a dimer (upper duplex band) (FIG. 3B). These results indicate that ALMC1 and ALMC2 AL amyloidosis cell lines synthesize and secrete large amount of IgG and λ light chains.

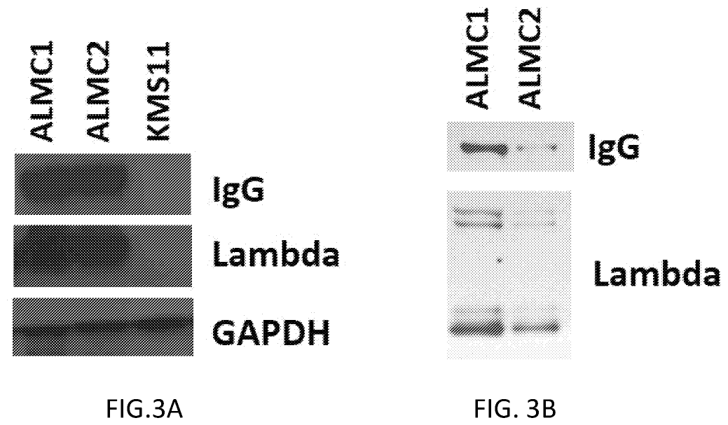

Furthermore, western blot was performed to detect the expression of SNAP 23 and SYNTAXIN-4. The expression of SNAP23 and SYNTAXIN-4 was detected in the whole cell lysate from ALMC1 and ALMC2 (FIG. 4). The results indicate that ALMC1 and ALMC2 Express High Level of SNAP 23 and SYNTAXIN-4 SNAREs.

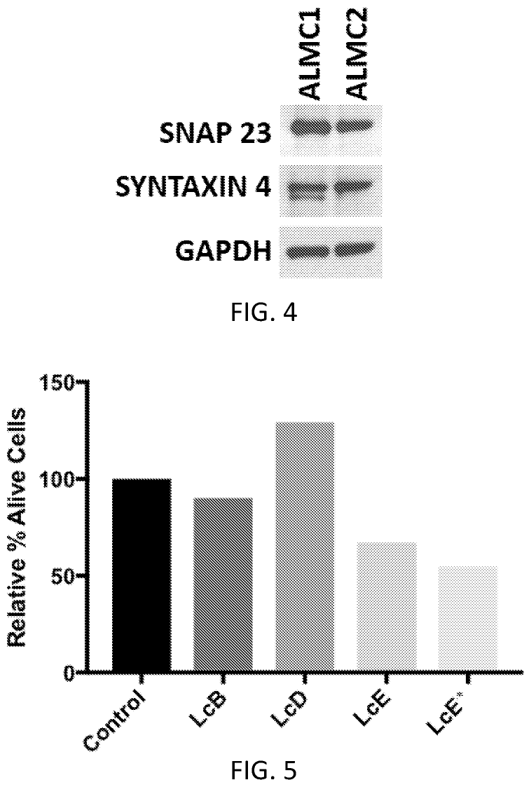

ALMC2 cells were then transduced with a lentiviral vector expressing different botulinum light chain serotypes (B, D, E and a mutant E comprising a K224D mutation) or an empty lentiviral vector (control) in frame with GFP. GFP positive cells were sorted 48 hours after transduction. Annexin V/7AAD staining was performed 72 hours post transduction to assess apoptosis. Histogram bars represent relative percentage of alive (Annexin V-/7AAD-) cells compared to control. LcE and mutant LcE serotypes resulted in significant loss of viability (FIG. 5). These results indicate that loss of viability upon expression of Bo light chain is serotype specific. Each Bo Lc has specificity for one or a few SNAREs. Therefore, results suggest that only targeting of certain SNARE proteins, but not others results in loss of viability. By using different Bo light chains with known SNARE specificity, we will be able to identify the SNAREs that are necessary to mediate cytotoxic phenotype.

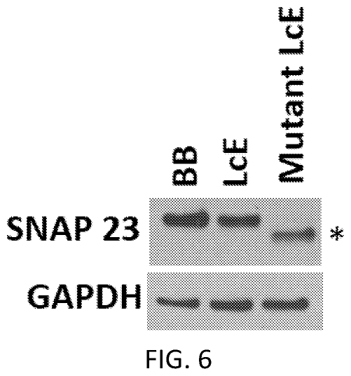

ALMC2 cells were transduced with a lentiviral vector expressing botulinum light chain serotype E, mutant E, or an empty lentiviral vector (control) in frame with GFP. GFP positive cells were sorted 48 hours after transduction and cells were harvested to obtain protein lysate. Western blot showed SNAP23 cleavage (asterisk) in cells transduced with mutant botulinum light chain serotype E, but not E (FIG. 6). The result is consistent with pattern of specificity for SNARE cleavage described above where mutant E, but not E is known to target SNAP23. Thus, expression of mutant Botulinum light chain E results in cleavage of SNAP23 which in turn leads to apoptosis.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Bianchi G, Anderson K C. Understanding biology to tackle the disease: Multiple myeloma from bench to bedside, and back. C A: a cancer journal for clinicians. 2014; 64:422-444.
2. Dispenzieri A, Merlini G. Immunoglobulin Light Chain Systemic Amyloidosis. Cancer treatment and research. 2016; 169:273-318.
3. Bridoux F, Leung N, Hutchison C A, et al. Diagnosis of monoclonal gammopathy of renal significance. Kidney international. 2015; 87:698-711.
4. Berenson J R, Anderson K C, Audell R A, et al. Monoclonal gammopathy of undetermined significance: a consensus statement. British journal of haematology. 2010; 150:28-38.
5. Rison R A, Beydoun S R. Paraproteinemic neuropathy: a practical review. BMC neurology. 2016; 16:13.
6. Kumar S K, Rajkumar S V, Dispenzieri A, et al. Improved survival in multiple myeloma and the impact of novel therapies. Blood. 2008; 111:2516-2520.
7. Kisselev A F, Goldberg A L. Proteasome inhibitors: from research tools to drug candidates. Chem Biol. 2001; 8:739-758.
8. Richardson P Q Sonneveld P, Schuster M W, et al. Bortezomib or high-dose dexamethasone for relapsed multiple myeloma. The New England journal of medicine. 2005; 352:2487-2498.
9. Bianchi G, Oliva L, Cascio P, et al. The proteasome load versus capacity balance determines apoptotic sensitivity of multiple myeloma cells to proteasome inhibition. Blood. 2009; 113:3040-3049.
10. Leung-Hagesteijn C, Erdmann N, Cheung G, et al. Xbpls-negative tumor B cells and pre-plasmablasts mediate therapeutic proteasome inhibitor resistance in multiple myeloma. Cancer cell. 2013; 24:289-304.
11. Oliva L, Orfanelli U, Resnati M, et al. The amyloidogenic light chain is a stressor that sensitizes plasma cells to proteasome inhibitor toxicity. Blood. 2017; 129:2132-2142.
12. Cenci S, Oliva L, Cerruti F, et al. Pivotal Advance: Protein synthesis modulates responsiveness of differentiating and malignant plasma cells to proteasome inhibitors. Journal of leukocyte biology. 2012; 92:921-931.
13. Hideshima T, Qi J, Paranal R M, et al. Discovery of selective small-molecule HDAC6 inhibitor for overcoming proteasome inhibitor resistance in multiple myeloma. Proceedings of the National Academy of Sciences of the United States of America. 2016; 113:13162-13167.
14. Gertz M A, Landau H, Comenzo R L, et al. First-in-Human Phase I/II Study of NEOD001 in Patients With Light Chain Amyloidosis and Persistent Organ Dysfunction. J Clin Oncol. 2016; 34:1097-1103.
15. Reales E, Mora-Lopez F, Rivas V, Garcia-Poley A, Brieva J A, Campos-Caro A. Identification of soluble N-ethylmaleimide-sensitive factor attachment protein receptor exocytotic machinery in human plasma cells: SNAP-23 is essential for antibody secretion. J Immunol. 2005; 175:6686-6693.
16. Gomez-Jaramillo L, Delgado-Perez L, Reales E, et al. Syntaxin-4 is implicated in the secretion of antibodies by human plasma cells. Journal of leukocyte biology. 2014; 95:305-312.
17. Gomez-Jaramillo L, Romero-Garcia R, Jimenez-Gomez G, et al. VAMP2 is implicated in the secretion of antibodies by human plasma cells and can be replaced by other synaptobrevins. Cellular & molecular immunology. 2016.
18. Shoemaker C B, Oyler G A. Persistence of Botulinum neurotoxin inactivation of nerve function. Curr Top Microbiol Immunol. 2013; 364:179-196.
19. Drexler H G Matsuo Y. Malignant hematopoietic cell lines: in vitro models for the study of multiple myeloma and plasma cell leukemia. Leukemia research. 2000; 24:681-703.
20. Chen S, Barbieri J T. Engineering botulinum neurotoxin to extend therapeutic intervention. Proceedings of the National Academy of Sciences of the United States of America. 2009; 106:9180-9184.
21. Masuyer Chaddock J A, Foster K A, Acharya K R. Engineered botulinum neurotoxins as new therapeutics. Annual review of pharmacology and toxicology. 2014; 54:27-51.
22. Bianchi G, Richardson P C; Anderson K C. Promising therapies in multiple myeloma. Blood. 2015; 126:300-310.

What is claimed is:

1. A method of treating a subject having a disorder associated with protein secretion, production, or deposition, that is pathogenic, the method comprising administering to the subject an effective amount of a composition comprising a Botulinum neurotoxin (BoNT) agent comprising a heavy chain and a light chain, wherein the BoNT inhibits the protein secretion, production, or deposition, that is pathogenic, thereby treating the disorder.

2. The method of claim 1, wherein the BoNT agent is a chimeric Botulinum neurotoxin.

3. The method of claim 2, wherein the chimeric BoNT agent targets plasma cells.

4. The method of claim 2, wherein the heavy chain of the chimeric BoNT agent targets one or more of markers selected from the group consisting of CD138, CD38, CD78, CD319, IL-6 receptor, and B-cell maturation antigen (BCMA).

5. The method of claim 2, wherein the light chain of the chimeric BoNT agent cleaves a soluble N-ethytmaleimide-sensitive factor attachment protein receptor (SNARE).

6. The method of claim 1, wherein the disorder is a plasma cell disorder.

7. The method of claim 6, wherein one or more plasma cells in the subject have an increased synthesis and/or secretion of paraprotein.

8. The method of claim 6, wherein one or more plasma cells in the subject have an increased synthesis and/or secretion of free light chains (FLC).

9. The method of claim 6, wherein the plasma disorder is multiple myeloma.

10. The method of claim 6, wherein the plasma disorder is Amyloid light-chain (AL) amyloidosis.

11. The method of claim 6, wherein the plasma cell disorder is monoclonal gammopathy of undermined significance (MGUS) or monoclonal gammopathy of renal significance (MGRS).

12. The method of claim 6, wherein the plasma cell disorder is paraproteinimic neuropathy.

13. The method of claim 6, wherein the plasma cell disorder is polyneuropathy, organomegaly, endocrinopathy monoclonal gammopathy and skin changes syndrome (POEMS).

14. The method of claim 1, wherein the disorder is non-AL amyloidosis.

15. The method of claim 1, wherein the disorder is a cancer whose pathogenic mechanism involves, or is due to, a secreted protein.

16. The method of claim 15, wherein the cancer is an insulinoma, a gastrinoma, a secreting adrenal tumor, an adenoma, a parathyroid adenoma, a pituitary adenoma, a carcinoid tumor, an adenocarcinoma, a pancreatic cancer, a breast cancer, an ovarian cancer or a colon cancer.

17. The method of claim 1, wherein the subject has a tumor characterized by high protein secretion.

18. The method of claim 17, wherein the tumor is an adenocarcinoma.

19. The method of claim 18, wherein the adenocarcinoma is of the pancreas, breast, or colon.

20. The method of any one of the preceding claims, wherein the subject is a human.

21. The method of any one of the preceding claims, wherein the subject is not subjected to chemotherapy.

22. The method of any one of the preceding claims, wherein the subject is also administered a proteasome inhibitor.

23. A method of treating a subject having a disorder associated with protein secretion, production, or deposition, that is pathogenic, the method comprising administering to the subject an effective amount of a composition comprising a nucleic acid that encodes a BoNT light chain.

24. The method of claim 23, wherein the BoNT light chain is a Botulinum E light chain.

25. The method of claim 23, wherein the BoNT light chain is a mutant Botulinum E light chain.

26. The method of any one of claims 23-25, wherein the nucleic acid is delivered by a lentiviral vector.

27. A pharmaceutical composition comprising a BoNT light chain and a proteasome inhibitor.

28. The pharmaceutical composition of claim 27, wherein the BoNT light chain is a Botulinum E light chain or a mutant Botulinum E light chain.

29. The pharmaceutical composition of claim 27 or 28, wherein the proteasome inhibitor is bortezomib, carfilzomib, ixazomib, marizomib (NPI-0052), peptide boronate (delanzomib), or epoxyketone (oprozimib).

30. A method of treating a subject having a disorder associated with protein secretion, production, or deposition, that is pathogenic, the method comprising administering to the subject an effective amount of a composition comprising a BoNT light no chain and a proteasome inhibitor.

31. A method of treating a subject having a disorder associated with protein secretion, production, or deposition, that is pathogenic, the method comprising administering to the subject an effective amount of a composition comprising a BoNT light chain.

32. The method of claim 31, wherein the BoNT light chain is a Botulinum E light chain or a mutant Botulinum E light chain.
